(19) United States
(12) Patent Application Publication
(10) Pub. No.: US 2016/0069967 A1
(43) Pub. Date: Mar. 10, 2016

(54) APPARATUS AND SYSTEM FOR IMAGING AN INTUBATED PATIENT

(71) Applicant: General Electric Company, Schenectady, NY (US)

(72) Inventors: Sung Man Moon, Florence, SC (US); Timothy John Havens, Florence, SC (US); Timothy John Bergfeld, Florence, SC (US)

(21) Appl. No.: 14/482,635

(22) Filed: Sep. 10, 2014

(51) Int. Cl. G01R 33/34; A61B 5/055

(52) U.S. Cl. CPC G01R 33/34; A61B 5/055

(57) ABSTRACT

The present application discloses a gradient coil apparatus for a Magnetic Resonance Imaging (MRI) system that comprises a cylindrical gradient coil assembly having a length along an axis and comprising an X-gradient coil, a Y-gradient coil and a Z-gradient coil. The gradient coil assembly further comprises an intubation channel, wherein the intubation channel extends radially from the axis and along at least a portion of the length.

### BACKGROUND OF THE INVENTION

The subject matter disclosed herein relates to Magnetic Resonance Imaging (MRI) and more specifically, a gradient coil for imaging an intubated patient.

Generally, the preferred position for a patient to undergo a MRI scan is centered in the magnet bore. However, this may be challenging when the patient, such as a neonate or infant is intubated. Currently, when imaging an intubated neonatal patient, the patient must be positioned below the iso-center of the magnet bore in order to accommodate the intubation equipment, such as tubing. As such, approximately one-third of the bore diameter is not utilized for imaging. This results in a lower image quality and does not allow the clinician to take advantage of the full imaging field of view. This necessitates the magnet bore having a larger than desired diameter and results in a more expensive MRI system.

Therefore, a gradient coil that accommodates for the intubation equipment connected to a neonatal patient is desired to increase image quality and decrease cost.

### BRIEF DESCRIPTION OF THE INVENTION

The above-mentioned shortcomings, disadvantages and problems are addressed herein which will be understood by reading and understanding the following specification.

In an embodiment, a gradient coil apparatus for a Magnetic Resonance Imaging (MRI) system comprises a cylindrical gradient coil assembly having a length along an axis and comprising an X-gradient coil, a Y-gradient coil and a Z-gradient coil. The gradient coil assembly further comprises an intubation channel, wherein the intubation channel extends radially from the axis and along at least a portion of the length.

In another embodiment, a gradient coil apparatus for a Magnetic Resonance Imaging (MRI) system comprises a gradient coil assembly having a length along an axis and comprising an X-gradient coil, a Y-gradient coil and a Z-gradient coil, wherein for at least a portion of the length the gradient coil assembly has a C-shaped cross-section perpendicular to the axis.

In another embodiment, a MRI system comprises a magnet configured to establish a magnetic field; a patient positioning area; and a gradient coil assembly adjacent the patient positioning area, the gradient coil assembly having an intubation channel.

In another embodiment, a gradient coil apparatus for a MRI system comprises a gradient coil assembly that is cylindrical along an axis and having a length along the axis, the gradient coil assembly comprising an X-gradient coil, a Y-gradient coil and a Z-gradient coil. The gradient coil assembly has a cross-section perpendicular to the axis comprising a continuous outer circumference and a discontinuous inner circumference, the gradient coil assembly having an intubation channel defined between the discontinuous portion of the inner circumference and the continuous outer circumference.

Various other features, objects, and advantages of the invention will be made apparent to those skilled in the art from the accompanying drawings and detailed description thereof.

### BRIEF DESCRIPTION OF THE DRAWINGS

FIG. 1 is a schematic block diagram of an exemplary magnetic resonance imaging (MRI) system in accordance with an embodiment of the disclosure;

FIG. 2 is a perspective view of a gradient coil assembly in accordance with a first embodiment of the disclosure;

FIG. 3 is a perspective view of a gradient coil assembly in accordance with a second embodiment of the disclosure;

FIG. 4 is a top view of the gradient coil assembly in accordance with the first embodiment of the disclosure;

FIG. 5 is a top view of the gradient coil assembly in accordance with the second embodiment of the disclosure;

FIG. 6 is a cross-sectional view of a gradient coil assembly in accordance with an embodiment of the disclosure;

FIG. 7 is a cross-sectional view of a gradient coil assembly in accordance with another embodiment of the disclosure;

FIG. 8 is a cross-sectional view of a gradient coil assembly in accordance with yet another embodiment of the disclosure; and

FIG. 9 is a cross-sectional view of a gradient coil assembly in accordance with another embodiment of the disclosure.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

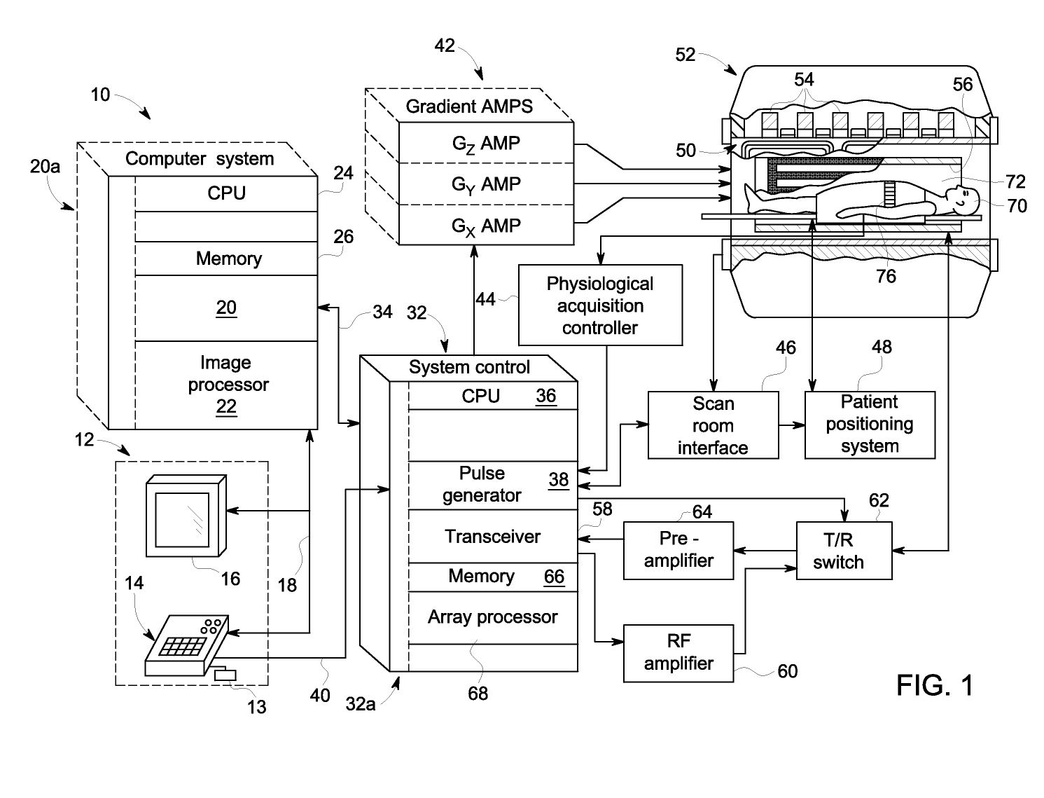

FIG. 1 is a schematic block diagram of an exemplary magnetic resonance imaging (MRI) system in accordance with an embodiment. The operation of MRI system 10 is controlled from an operator console 12 that includes a keyboard or other input device 13, a control panel 14, and a display 16. The console 12 communicates through a link 18 with a computer system 20 and provides an interface for an operator to prescribe MRI scans, display resultant images, perform image processing on the images, and archive data and images. The computer system 20 includes a number of modules that communicate with each other through electrical and/or data connections, for example, such as are provided by using a backplane 20a. Data connections may be direct wired links or may be fiber optic connections or wireless communication links or the like. The modules of the computer system 20 include an image processor module 22, a CPU module 24 and a memory module 26 which may include a frame buffer for storing image data arrays. In an alternative embodiment, the image processor module 22 may be replaced by image processing functionality on the CPU module 24. The computer system 20 is linked to archival media devices, permanent or back-up memory storage or a network. Computer system 20 may also communicate with a separate system control computer 32 through a link 34. The input device 13 can include a mouse, joystick, keyboard, track ball, touch activated screen, light wand, voice control, or any similar or equivalent input device, and may be used for interactive geometry prescription.

The system control computer 32 includes a set of modules in communication with each other via electrical and/or data connections 32a. Data connections 32a may be direct wired links, or may be fiber optic connections or wireless communication links or the like. In alternative embodiments, the modules of computer system 20 and system control computer 32 may be implemented on the same computer system or a plurality of computer systems. The modules of system control computer 32 include a CPU module 36 and a pulse generator module 38 that connects to the operator console 12 through a communications link 40. The pulse generator module 38 may alternatively be integrated into the scanner equipment (e.g., resonance assembly 52). It is through link 40 that the system control computer 32 receives commands from the operator to indicate the scan sequence that is to be performed. The pulse generator module 38 operates the system components that play out (i.e., perform) the desired pulse sequence by sending instructions, commands and/or requests describing the timing, strength and shape of the RF pulses and pulse sequences to be produced and the timing and length of the data acquisition window. The pulse generator module 38 connects to a gradient amplifier system 42 and produces data called gradient waveforms that control the timing and shape of the gradient pulses that are to be used during the scan. The pulse generator module 38 may also receive patient data from a physiological acquisition controller 44 that receives signals from a number of different sensors connected to the patient, such as ECG signals from electrodes attached to the patient. The pulse generator module 38 connects to a scan room interface circuit 46 that receives signals from various sensors associated with the condition of the patient and the magnet system. It is also through the scan room interface circuit 46 that a patient positioning system 48 receives commands to move the patient table to the desired position for the scan.

The gradient waveforms produced by the pulse generator module 38 are applied to gradient amplifier system 42 which is comprised of Gₓ, G_{y} and G_{z} amplifiers. Each gradient amplifier excites a corresponding physical gradient coil in a gradient coil assembly generally designated 50 to produce the magnetic field gradient pulses used for spatially encoding acquired signals. The gradient coil assembly 50 forms part of a resonance assembly 52 that includes a polarizing superconducting magnet with superconducting main coils 54. Resonance assembly 52 may include a whole-body RF coil 56, surface or parallel imaging coils 76 or both. The coils 56, 76 of the RF coil assembly may be configured for both transmitting and receiving or for transmit-only or receive-only. A patient or imaging subject 70 may be positioned within a cylindrical patient imaging volume 72 of the resonance assembly 52. A transceiver module 58 in the system control computer 32 produces pulses that are amplified by an RF amplifier 60 and coupled to the RF coils 56, 76 by a transmit/receive switch 62. The resulting signals emitted by the excited nuclei in the patient may be sensed by the same RF coil 56 and coupled through the transmit/receive switch 62 to a preamplifier 64. Alternatively, the signals emitted by the excited nuclei may be sensed by separate receive coils such as parallel coils or surface coils 76. The amplified MR signals are demodulated, filtered and digitized in the receiver section of the transceiver 58. The transmit/receive switch 62 is controlled by a signal from the pulse generator module 38 to electrically connect the RF amplifier 60 to the RF coil 56 during the transmit mode and to connect the preamplifier 64 to the RF coil 56 during the receive mode. The transmit/receive switch 62 can also enable a separate RF coil (for example, a parallel or surface coil 76) to be used in either the transmit or receive mode.

The MR signals sensed by the RF coil 56 or parallel or surface coil 76 are digitized by the transceiver module 58 and transferred to a memory module 66 in the system control computer 32. Typically, frames of data corresponding to MR signals are stored temporarily in the memory module 66 until they are subsequently transformed to create images. An array processor 68 uses a known transformation method, most commonly a Fourier transform, to create images from the MR signals. These images are communicated through the link 34 to the computer system 20 where it is stored in memory. In response to commands received from the operator console 12, this image data may be archived in long-term storage or it may be further processed by the image processor 22 and conveyed to the operator console 12 and presented on display 16.

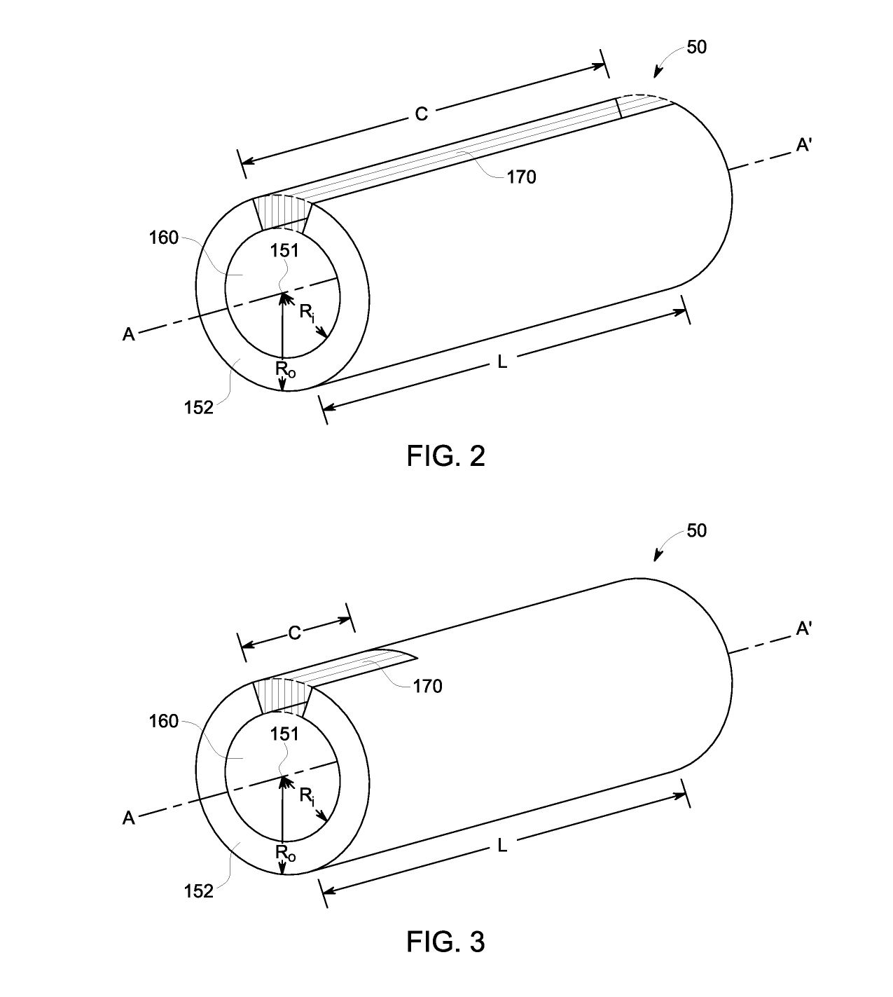

Referring to FIG. 2, a perspective view of the gradient coil assembly 50 is shown in accordance with an embodiment of the disclosure. Gradient coil assembly 50 is substantially cylindrical in shape, defined by a length L and an outer radius Rₒ. An axis A-A′ extends through an iso-center 151 of the gradient coil assembly 50.

Gradient coil assembly 50 comprises a plurality of gradient coils 152. The outer radius Rₒ extends from the iso-center 151 to the outer side of the plurality of gradient coils 152. An inner radius Rᵢ extends from the iso-center 151 to the inner side of the plurality of gradient coils 152. In this embodiment, inner radius Rᵢ is less than outer radius Rₒ.

Gradient coil assembly 50 comprises a hollow bore 160. The hollow bore 160 may be configured to comprise a patient positioning area that is able to accommodate a patient table and patient. The patient will hereinafter be described as a neonate or infant. It should be appreciated, however, that other age and/or size patient demographics may be envisioned within the scope of this disclosure. The hollow bore 160 extends along axis A-A′ and is bounded by inner radius R.

The gradient coil assembly 50 may include an intubation channel 170. The intubation channel 170 is configured to accommodate the intubation and or ventilation equipment associated with a patient (not shown). The intubation equipment may include but not be limited to tubing.

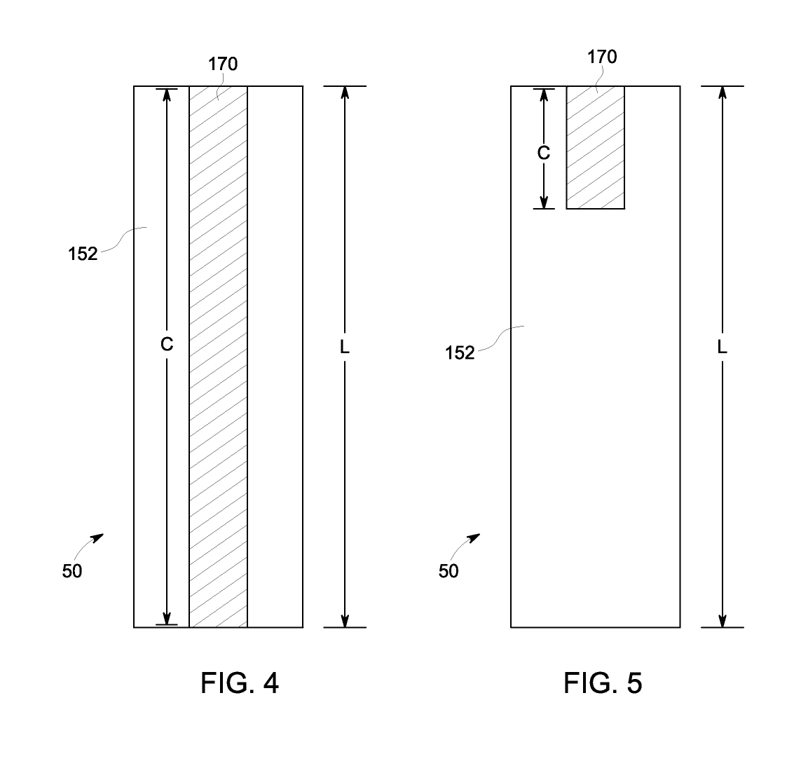

As depicted in FIGS. 2 and 3, the intubation channel 170 is the cross-hatched volume bounded between Rᵢ and Rₒ and extending for a length C of the gradient coil assembly 50. In FIGS. 4 and 5, top views of the gradient coil assembly 50 are shown in accordance with two embodiments. In these figures as well, the intubation channel 170 is depicted by cross-hatching.

In the embodiment shown in FIGS. 2 and 4, the intubation channel 170 extends substantially along the entire the length L of the gradient coil assembly 50. In this embodiment, length C of the intubation channel 170 is substantially equal to length L of the gradient coil assembly 50. It should be appreciated, however, that various lengths C of the intubation channel 170 may be envisioned. For example, as depicted in FIGS. 3 and 5, the intubation channel 170 may extend for a portion of length L of the gradient coil assembly 50. In this embodiment, length C of the intubation channel 170 is less than length L of the gradient coil assembly 50.

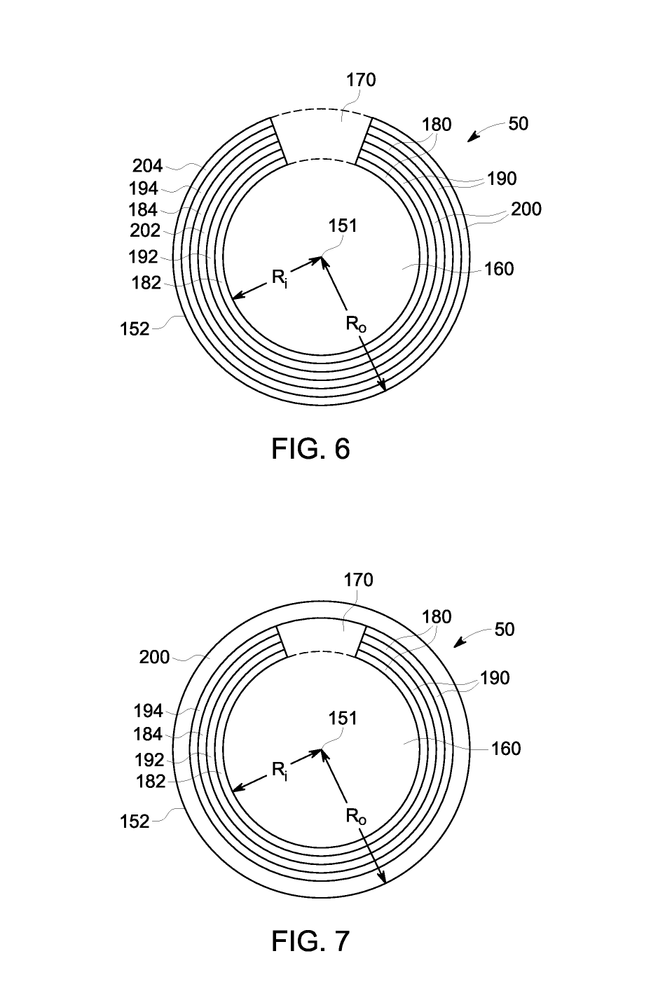

Referring to FIG. 6, a cross-sectional view of the gradient coil assembly 50 perpendicular to axis A-A′ is shown in accordance with an embodiment. The gradient coil assembly 50 comprises the plurality of gradient coils 152. The plurality of gradient coils 152 may comprise an X-gradient coil 180, a Y-gradient coil 190 and a Z-gradient coil 200. The X-gradient coil 180 may comprise an inner, primary layer 182 and an outer, shielding layer 184. The Y-gradient coil 190 may comprise an inner, primary layer 192 and an outer, shielding layer 194. The Z-gradient coil 200 may comprise an inner, primary layer 202 and an outer, shielding layer 204. The plurality of gradient coils 152 comprises an inner circumference related to inner radius Rᵢ and an outer circumference related to Rₒ.

In the depicted embodiment, the gradient coil assembly 50 comprises the intubation channel 170. Intubation channel 170 is the area bounded between the inner radius Ri and the outer radius Ro, extending radially through the X-gradient coil 180, the Y-gradient coil 190 and the Z-gradient coil 200. In this embodiment, both the inner circumference and the outer circumference of the plurality of gradient coils 152 are discontinuous, and the cross-section of the gradient coil assembly 50 is substantially C-shaped.

Referring to FIG. 7, a cross-sectional view of the gradient coil assembly 50 is shown in accordance with another embodiment. Similar to the embodiment depicted in FIG. 6, the plurality of gradient coils 152 comprises the X-gradient coil 180, the Y-gradient coil 190 and the Z-gradient coil 200. The X-gradient coil 180 may comprise the inner, primary layer 182 and the outer, shielding layer 184. The Y-gradient coil 190 may comprise the inner, primary layer 192 and the outer, shielding layer 194. The gradient coil assembly 50 comprises intubation channel 170. In this embodiment, the intubation channel 170 extends radially through the x-gradient coil 180 and the Y-gradient coil assembly, but does not extend through the z-gradient coil 200. Therefore, the inner circumference of the plurality of gradient coils 152 is discontinuous while the outer circumference of the plurality of the gradient coils 152 is continuous. The continuity of the outer circumference is configured to strengthen the overall structure of the gradient coil assembly 50 and further improve image quality.

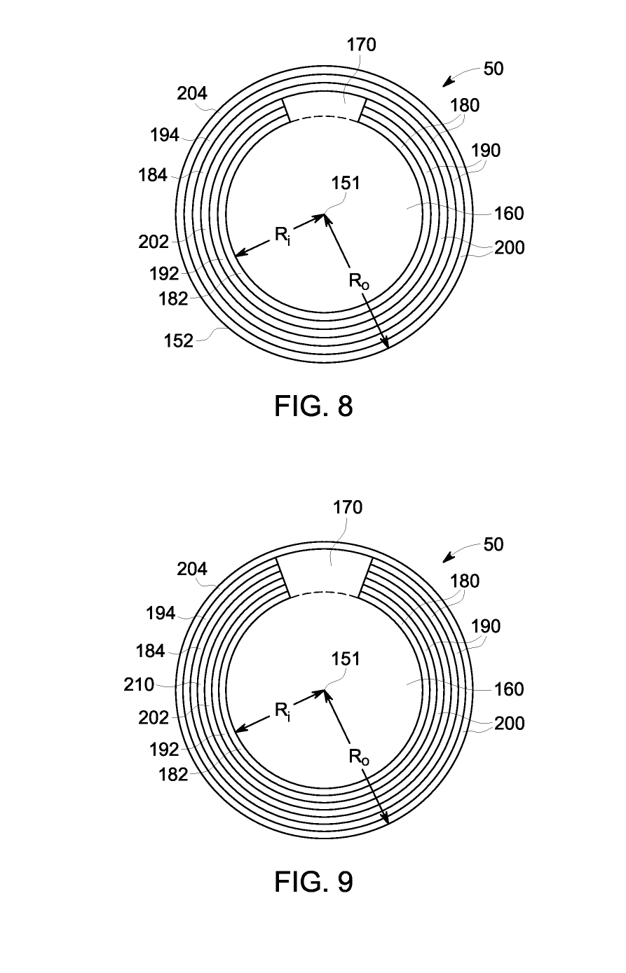

Referring to FIG. 8, a cross-sectional view of the gradient coil assembly 50 is shown in accordance with yet another embodiment. The plurality of gradient coils 152 comprises the X-gradient coil 180, the Y-gradient coil 190 and the Z-gradient coil 200. The X-gradient coil 180 may comprise the inner, primary layer 182 and the outer, shielding layer 184. The Y-gradient coil 190 may comprise the inner, primary layer 192 and the outer, shielding layer 194. The Z-gradient coil 200 may comprise the inner, primary layer 202 and the outer, shielding layer 204. As shown in the embodiment depicted in FIG. 8, the intubation channel 170 extends radially through the primary layers 182, 192, 202 but not through the shielding layers 184, 194, 204. In this embodiment, the inner circumference of the plurality of gradient coils 152 is discontinuous while the other circumference of the plurality of gradient foils 152 is continuous. The continuity of the outer circumference is configured to strengthen the overall structure of the gradient coil assembly 50 and further improve image quality.

Referring to FIG. 9, a cross-sectional view of the gradient coil assembly 50 is shown in accordance with another embodiment. The plurality of gradient coils 152 comprises the X-gradient coil 180, the Y-gradient coil 190 and the Z-gradient coil 200. The X-gradient coil 180 comprises the inner, primary layer 182 and the outer, shielding layer 184. The Y-gradient coil 190 comprises the inner, primary layer 192 and the outer, shielding layer 194. The Z-gradient coil 200 comprises the inner, primary layer 202 and an outer, shielding layer 204. The gradient coil assembly 50 may also comprise a separation layer 210. The separation layer 210 may comprise cooling materials, shimming materials, or a combination thereof. As shown in the embodiment depicted in FIG. 9, the intubation channel 170 extends radially through the primary layers 182, 192, 202, the separation layer 210 and shielding layers 184 and 194, but the intubation does not extend through the shielding layer 204. The continuity of the shielding layer 204 is configured to strengthen the overall structure of the gradient coil assembly 50 and further improve image quality.

It should be appreciated that various other embodiments of the intubation channel 170 may be envisioned within the scope of this disclosure. For example, the intubation channel may not be uniformly sized and/or shaped along length C.

It should also be appreciated that the intubation channel 170 of the gradient coil assembly 50 may be formed in various ways. For example, the gradient coils 180, 190 and 200 may comprise finger-print patterns similar to a planar gradient coil known in the art, and the intubation channel 170 may be formed by bending the gradient coils 180, 190, 200 about axis A-A′, but not joining the ends of at least one of gradient coils 180, 190, 200 in a C-shaped cross-section. In another example, the intubation channel 170 may be formed by rotating X-gradient coil 180 and the Y-gradient coil 190 from their original axes. In yet another example, the traditional finger-print pattern can be split by half creating a gap in the middle of the pattern. This results in having three or four finger-print patterns instead of two as in the traditional gradient coil finger-print pattern design.

A gradient coil assembly 50 comprising the intubation channel 170 provides numerous benefits to clinicians and patients. The intubation channel 170 provides users easier access for positioning neonatal patients in the bore 160 by allowing more room for intubation equipment. The intubation channel 170 also increases patient safety by decreasing potential CO₂ build-up as the intubation channel 170 allows for increased air flow through the bore 160 and provides a path for CO₂ to exit the bore 160. Accommodating intubation equipment in the intubation channel 170 instead of the bore 160 allows for a reduction in Rᵢ and bore size, as well as by as much as 5cm in magnet size. A smaller magnet results in increased image quality, reduced stray field is both radial and axial directions, and reduced system cost.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A gradient coil apparatus for a Magnetic Resonance Imaging (MRI) system, comprising:
a cylindrical gradient coil assembly having a length along an axis and comprising an X-gradient coil, a Y-gradient coil and a Z-gradient coil;
the gradient coil assembly further comprising an intubation channel, wherein the intubation channel extends radially from the axis and along at least a portion of the length.

2. The gradient coil apparatus of claim 1, wherein the intubation channel extends along the entire length of the gradient coil assembly.

3. The gradient coil apparatus of claim 1, wherein the intubation channel extends along only a portion of the length of the gradient coil assembly.

4. The gradient coil apparatus of claim 1, wherein the intubation channel extends radially through the X-gradient coil and the Y-gradient coil.

5. The gradient coil apparatus of claim 4, wherein the Z-gradient coil comprises a primary layer and a shielding layer and wherein the intubation channel extends radially through the primary layer but not through the shielding layer.

6. The gradient coil apparatus of claim 1, wherein the intubation channel extends radially through the X-gradient coil, the Y-gradient coil and the Z-gradient coil.

7. The gradient coil apparatus of claim 1, wherein the X-gradient coil, Y-gradient coil and the Z-gradient coil each comprise a primary layer and a shielding layer and wherein the intubation channel extends radially through the primary layers but not through the shielding layers.

8. The gradient coil apparatus of claim 1, wherein the gradient coil assembly is sized for neonatal imaging.

9. A gradient coil apparatus for a Magnetic Resonance Imaging (MRI) system, comprising:
a gradient coil assembly having a length along an axis and comprising an X-gradient coil, a Y-gradient coil and a Z-gradient coil, wherein for at least a portion of the length the gradient coil assembly has a C-shaped cross-section perpendicular to the axis.

10. The gradient coil apparatus of claim 9, wherein the C-shaped cross-section extends along the entire length of the gradient coil assembly.

11. The gradient coil apparatus of claim 9, wherein the gradient coil assembly is sized for neonatal imaging.

12. A Magnetic Resonance Imaging (MRI) system, comprising:
a magnet configured to establish a magnetic field;
a patient positioning area; and
a gradient coil assembly adjacent the patient positioning area, the gradient coil assembly having an intubation channel.

13. The MRI system of claim 12, wherein the gradient coil assembly has a length along an axis and comprises an X-gradient coil, a Y-gradient coil and a Z-gradient coil, and the intubation channel extends along at least a portion of the length.

14. The MRI system of claim 12, wherein the intubation channel extends along the entire length of the gradient coil assembly.

15. The MRI system of claim 12, wherein the intubation channel extends radially through the X-gradient coil and the Y-gradient coil.

16. The MRI system of claim 12, wherein the intubation channel extends radially through the X-gradient coil, the Y-gradient coil and the Z-gradient coil.

17. The MRI system of claim 12, wherein the X-gradient coil, Y-gradient coil and the Z-gradient coil each comprise a primary layer and a shielding layer and wherein the intubation channel extends radially through the primary layers but not through the shielding layers.

18. The MRI system of claim 12, wherein the gradient coil assembly is sized for neonatal imaging.

19. A gradient coil apparatus for a Magnetic Resonance Imaging (MRI) system, comprising:
a gradient coil assembly that is cylindrical along an axis and having a length along the axis, the gradient coil assembly comprising an X-gradient coil, a Y-gradient coil and a Z-gradient coil;
wherein the gradient coil assembly has a cross-section perpendicular to the axis comprising a continuous outer circumference and a discontinuous inner circumference, the gradient coil assembly having an intubation channel defined between the discontinuous portion of the inner circumference and the continuous outer circumference.

20. The gradient coil apparatus of claim 19, wherein the gradient coil assembly is sized for neonatal imaging.
